(19) United States
(12) Patent Application Publication
(10) Pub. No.: US 2006/0211710 A1
(43) Pub. Date: Sep. 21, 2006

(54) Substituted aryl 1,4-pyrazine derivatives

(75) Inventors: Patrick R. Verhoest, Old Lyme, CT (US); Robert L. Hoffmann, San Marcos, CA (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/363,009

(22) Filed: Feb. 27, 2006

Related U.S. Application Data
(60) Provisional application No. 60/662,917, filed on Mar. 17, 2005.

(51) Int. Cl. A61K 31/496; C07D 403/02

(52) U.S. Cl. 514/255.05; 544/405

(57) ABSTRACT

The invention is directed to compounds of Formula I, described herein, as well as pharmaceutically acceptable salts thereof, which act as CRF₁ antagonists and are useful in the treatment of disorders and diseases associated with CRF₁ receptors, including CNS-related disorders and diseases.

### BACKGROUND OF THE INVENTION

This invention relates to substituted aryl 1,4-pyrazine derivatives and processes for preparing them, pharmaceutical compositions containing them, and methods of using them to treat a disorder or condition which can be effected or facilitated by antagonizing a CRF receptor, including but not limited to disorders induced or facilitated by CRF, such as anxiety disorders, and depression and stress related disorders. Additionally this invention relates to the use of such compounds as probes for the localization of CRF₁ receptors in cells or tissues.

Corticotropin releasing factor (CRF) is a 41 amino acid peptide that is the primary physiological regulator of proopiomelanocortin (POMC) derived peptide secretion from the anterior pituitary gland [J. Rivier et al., Proc. Natl. Acad. Sci (USA) 80:4851 (1983); W. Vale et al., Science 213:1394 (1981)]. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in the brain [W. Vale et al., Rec. Prog. Horm. Res. 39:245 (1983); F. Koob, Persp. Behav. Med. 2:39 (1985); E. B. De Souza et al., J. Neurosci. 5:3189 (1985)]. There is also evidence that CRF plays a significant role in integrating the response in the immune system to physiological, psychological, and immunological stressors [J. E. Blalock, Physiological Reviews 69:1 (1989); J. E. Morley, Life Sci. 41:527 (1987)].

There is evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis, as they relate to the dysfunction of CRF neurons in the central nervous system [for a review, see: E. B. De Souze, Hosp. Practice 23:59 (1988)].

Anxiety disorders are a group of diseases, recognized in the art, that includes phobic disorders, anxiety states, post-traumatic stress disorder and atypical anxiety disorders [The Merck Manual of Diagnosis and Therapy, 16^{th} edition (1992)]. Emotional stress is often a precipitating factor in anxiety disorders, and such disorders generally respond to medications that lower response to stress.

In affective disorder, or major depression, the concentration of CRF is significantly increased in the cerebral spinal fluid (CSF) of drug-free individuals [C. B. Nemeroff et al., Science 226:1342 (1984); C. M. Banki et al., Am. J. Psychiatry 144:873 (1987); R. D. France et al., Biol. Psychiatry 28:86 (1988); M. Arato et al., Biol. Psychiatry 25:355 (1989)]. Furthermore, the density of CRF receptors is significantly decreased in the frontal cortex of suicide victims, consistent with a hypersecretion of CRF [C. B. Memeroff et al., Arch. Gen. Psychiatry 45:577 (1988)]. In addition, there is a blunted adrenocorticotropin (ACTH) response to CRF (i.v. administered) observed in depressed patients [P. W. Gold et al., Am. J. Psychiatry 141:619 (1984); F. Holsboer et al., Psychoneuroendocrinology 9:147 (1984); P. W. Gold et al., New Engl. J. Med. 314:1129 (1986)]. Preclinical studies in rats and non-human primates provide additional support for the hypothesis that hypersecretion of CRF may be involved in the symptoms seen in human depression [R. M. Sapolsky, Arch. Gen. Psychiatry 46:1047 (1989)]. There is also preliminary evidence that tricyclic antidepressants can alter CRF levels and thus modulate the numbers of receptors in the brain [Grigoriadis et al., Neuropsychopharmacology 2:53 (1989)].

CRF has also been implicated in the etiology of anxiety-related disorders, and is known to produce anxiogenic effects in animals. Interactions between benzodiazepine/non-benzodiazepine anxiolytics and CRF have been demonstrated in a variety of behavioral anxiety models [D. R. Britton et al., Life Sci. 31:363 (1982); C. W. Berridge and A. J. Dunn Regul. Peptides 16:83 (1986)]. Preliminary studies using the putative CRF receptor antagonist α-helical ovine CRF (9-41) in a variety of behavioral paradigms demonstrates that the antagonist produces “anxiolytic-like” effects that are qualitatively similar to the benzodiazepines [C. W. Berridge and A. J. Dunn Horm. Behav. 21:393 (1987), Brain Research Reviews 15:71 (1990)].

Neurochemical, endocrine and receptor binding studies have all demonstrated interactions between CRF and benzodiazepine anxiolytics, providing further evidence for the involvement of CRF in these disorders. Chlodiazepoxide attenuates the “anxiogenic” effects of CRF both in the conflict test [K. T. Britton et al., Psychopharmacology 86:170 (1985); K. T. Britton et al., Psychopharmacology 94:306 (1988)] and in the acoustic startle test [N. R. Swerdlow et al., Psychopharmacology 88:147 (1986)] in rats. The benzodiazepine receptor antagonist Ro 15-1788, which was without behavioral activity alone in the operant conflict test, reversed the effects of CRF in a dose-dependent manner while the benzodiazepine inverse agonist FG 7142 enhanced the actions of CRF [K. T. Britton et al., Psychopharmacology 94:396 (1988)]. The mechanisms and sites of action through which conventional anxiolytics and antidepressants produce their therapeutic effects remain to be elucidated. Preliminary studies, examining the effects of a CRF₁ receptor antagonist peptide (═-helical CRF₉₋₄₁) in a variety of behavioral paradigms, have demonstrated that the CRF₁ antagonist produces “anxiolytic-like” effects qualitatively similar to the benzodiazepines [for a review, see: G. F. Koob and K. T. Britton, In: Corticotropin-Releasing Factor: Basic and Clinical Studies of a Neuropeptide, E. B. De Souza and C. B. Nemeroff eds., CRC Press p.221 (1990)].

The use of CRF₁ antagonists for the treatment of Syndrome X has also been described in U.S. patent application Ser. No. 09/696,822, filed Oct. 26, 2000, now issued as U.S. Pat. No. 6,589,947and European Patent Application No. 003094414, filed Oct. 26, 2000, which are also incorporated in their entireties herein by reference. Methods for using CRF₁ antagonists to treat congestive heart failure are described in U.S. Ser. No. 09/248,073, filed Feb. 10, 1999, now U.S. Pat. No. 6,043,260 (March 28, 2000) which is also incorporated herein in its entirety by reference.

CRF is known to have a broad extrahypothalmic distribution in the CNS, contributing therein to a wide spectrum of autonomic behavioral and physiological effects [see, e.g., Vale et al., 1983; Koob, 985; and E. B. De Souze et al., 1985]. For example, CRF concentrations are significantly increased in the cerebral spinal fluid of patients afflicted with affective disorder or major depression [see, e.g., Nemeroff et al., 1984; Banki et al., 1987; France et al., 1988; Arato et al., 1989]. Moreover, excessive levels of CRF are known to produce anxiogenic effects in animal models [see, e.g., Britton et al., 1982; Berridge and Dunn, 1986 and 1987], and CRF₁ antagonists are known to produce anxiolytic effects; accordingly, therapeutically effective amounts of compounds provided herein are, for example, determined by assessing the anxiolytic effects of varying amounts of the compounds in such animal models.

The following patents or patent applications disclose compounds as antagonists of CRF₁ receptors: WO01/60806, WO97/35901, WO98/29119, W097/36886, W097/36898, and U.S. Pat. Nos. 5,872,136, 5,880,140, and 5,883,105. The compounds are useful for treating CNS-related disorders, particularly affective disorders and acute and chronic neurological disorders.

U.S. Patent publication 2003-0144297, incorporated herein in its entirety by reference, also discloses compounds as antagonists of CRF.

### SUMMARY OF THE INVENTION

We have found that compounds of Formula I, described below, as well as pharmaceutically acceptable salts thereof, are CRF₁ antagonists and are useful in the treatment of disorders and diseases associated with CRF₁ receptors, including CNS-related disorders and diseases.

Thus, this invention provides a compound of Formula I, 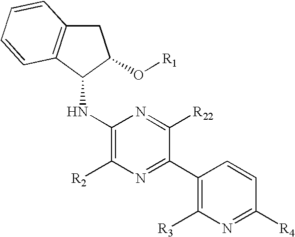
or a pharmaceutically acceptable salt thereof, wherein

R₁ is C₁-C₆ alkyl, C₁-C₆ alkenyl, C₁-C₆ alkynyl, C(O)C₁-C₆ alkyl, C(O)C_{-C}₆ alkenyl or C(O)C₁-C₆ alkynyl;

R₂ is C₁-C₆ alkyl, C₁-C₆ alkenyl, or C₁-C₆ alkynyl;

R₂₂ is C₁-C₆ alkyl, C₁-C₆ alkenyl, or C₁-C₆ alkynyl;

R₃ is C₁-C₆ alkyl, C₁-C₆ alkenyl, C₁-C₆ alkynyl, halogen, OC₁-C₆ alkyl, O₁-C₆ alkenyl, or O₁-C₆ alkynyl;

R₄ is C₁-C₆ alkyl, C₁-C₆ alkenyl, C₁-C₆ alkynyl, halogen, OC₁-C₆ alkyl, OC₁-C₁ alkenyl, OC₁-C₆ alkynyl or NR₅R₆;

R₅ is hydrogen, C₁-C₆ alkyl, C₁-C₆ alkenyl, or C₁-C₆ alkynyl;

and

R₆ is hydrogen, C₁-C₆ alkyl, C₁-C₆ alkenyl, or C₁-C₆ alkynyl.

In another aspect, the present invention provides a method for the treatment of a disorder or disease that is associated with CRF₁ receptors, or a disorder the treatment of which can be effected or facilitated by antagonizing CRF₁ in a mammal, particularly in a human, such as generalized anxiety disorder, social anxiety disorder; panic disorder; obsessive-compulsive disorder; anxiety with co-morbid depressive illness; affective disorder; anxiety; eating disorders; and depression, the method comprising administering to the mammal the compound of formula I.

In another aspect, the present invention provides a pharmaceutical composition comprising a pharmaceutically acceptable carrier or excipient and a compound of the invention. The compound of the invention in the composition may be present in an amount that is therapeutically effective for the treatment of a disorder or disease that is associated with CRF₁ receptors, or a disorder the treatment of which can be effected or facilitated by antagonizing CRF₁, in a mammal, particularly in a human.

In another aspect, the present invention provides a method of treating a disorder manifesting hypersecretion of CRF in a mammal, comprising administering to the mammal a therapeutically effective amount of a compound of the invention.

Preferably, the mammal is a mammal in need of the treatment described herein.

In another aspect, the present invention provides a method for screening for ligands for CRF₁ receptors, which method comprises: a) carrying out a competitive binding assay with CRF₁ receptors, a compound of the invention which is labeled with a detectable label, and a candidate ligand; and b) determining the ability of said candidate ligand to displace said labeled compound.

In another aspect, the present invention provides a method for detecting CRF receptors in tissue comprising: a) contacting a compound of the invention which is labeled with a detectable label, with a tissue, under conditions that permit binding of the compound to the tissue; and b) detecting the labeled compound bound to the tissue.

In another aspect, the present invention provides a method of inhibiting the binding of CRF to a CRF₁ receptor, comprising contacting a compound of the invention with a solution comprising cells expressing the CRF₁ receptor, wherein the compound is present in the solution at a concentration sufficient to inhibit the binding of CRF to the CRF₁ receptor.

In another aspect, the present invention provides a method of reducing the level of CRF binding in vitro to cells expressing the CRF₁ receptor, comprising contacting a compound according to claim 1 with a solution comprising the cells, wherein the compound is present in the solution at a concentration sufficient to reduce levels of CRF binding to the cells in vitro.

In another aspect, the present invention provides an article of manufacture comprising: a) a packaging material; b) a compound of the invention; and c) a label or package insert contained within said packaging material indicating that said compound is effective for treating a a disorder or disease that is associated with CRF₁ receptors, or a disorder the treatment of which can be effected or facilitated by antagonizing CRF₁, in a mammal.

In still another aspect, the present invention provides for the use of a compound of the invention in a binding assay, wherein one or more of the compounds may be joined to a label, where the label can directly or indirectly provide a detectable signal. Various labels include radioisotopes, fluorescers, chemiluminescers, specific binding molecules, particles, e.g. magnetic particles, and the like.

In yet another aspect, the present invention relates to the use of the compounds of the invention (particularly labeled compounds of this invention) as probes for the localization of receptors in cells and tissues and as standards and reagents for use in determining the receptor-binding characteristics of test compounds.

Exemplary embodiments of the invention include compounds of formula I in which R₁ is ethyl or C(O)CH₃.

Exemplary embodiments of the invention also include compounds of formula I in which R₂ is ethyl and R₂₂ is ethyl.

Exemplary embodiments of the invention also include compounds of formula I in which R₃ is C₁-C₆ alkyl, C₁-C₆ alkenyl, or C₁-C₆ alkynyl.

Exemplary embodiments of the invention also include compounds of formula I in which R₄ is NR₅R₆.

Exemplary embodiments of the invention also include compounds of formula I in which R₃ is C₁-C₆ alkyl, C₁-C₆ alkenyl, or C₁-C₆ alkynyl and R₄ is NR₅R₆.

Exemplary embodiments of the invention also include compounds of formula I in which R₃ is methyl and R₄ is N(CH₃)₂.

A compound of the invention may show advantageous solubility in water and gastric fluids. As an example, a compound of the invention where R₄ is NR₅R₆ may show advantageous solubility in water and gastric fluids. As another example, a compound of the invention where R₃ is C₁-C₆ alkyl and R₄ is NR₅R₆ may show advantageous solubility in water and gastric fluids. In a further exemplary embodiment, a compound of the invention where R₃ is methyl and R₄ is N(CH₃)₂ may show advantageous solubility in water and gastric fluids.

As used herein, “halogen” is a group selected from —F, —Cl, —Br, and —I.

As used herein, the term “C₁-C₆ alkyl” means both straight and branched chain saturated moieties having from 1-6 carbon atoms.

As used herein, the term “C₁-C₆ alkenyl” means both straight and branched chain moieties having from 1-6 carbon atoms containing one or more double bonds.

As used herein, the term “C₁-C₆ alkynyl” means both straight and branched chain moieties having from 1-6 carbon atoms containing one or more triple bonds.

As used herein, the term “pharmaceutically acceptable salt” refers to a salt prepared from pharmaceutically acceptable non-toxic acids, including inorganic acids and organic acids. Suitable non-toxic acids include inorganic and organic acids of basic residues such as amines, for example, acetic, benzenesulfonic, benzoic, amphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, barbaric acid, p-toluenesulfonic and the like; and alkali or organic salts of acidic residues such as carboxylic acids, for example, alkali and alkaline earth metal salts derived from the following bases: sodium hydride, sodium hydroxide, potassium hydroxide, calcium hydroxide, aluminum hydroxide, lithium hydroxide, magnesium hydroxide, zinc hydroxide, ammonia, trimethylammonia, triethylammonia, ethylenediamine, lysine, arginine, ornithine, choline, N,N″-dibenzylethylenediamine, chloroprocaine, diethanolamine, procaine, n-benzylphenethylamine, diethylamine, piperazine, tris(hydroxymethyl)-aminomethane, tetramethylammonium hydroxide, and the like. Pharmaceutically acceptable salts of the compounds of Formula I can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ea., Mack Publishing Company, Easton, Pa., 1985, p. 1418, the disclosure of which is hereby incorporated by reference.

In an exemplary embodiment, the salt of a compound of formula I and p-toluenesulfonic acid is a pharmaceutically acceptable salt of a compound of formula I.

The term “therapeutically effective amount” of a compound of this invention means an amount effective to antagonize abnormal level of CRF or treat the symptoms of affective disorder, anxiety, depression, or other disorders described herein above, in a host.

The term “compound of the invention” means a compound of Formula I or a pharmaceutically acceptable salt thereof.

The claimed invention also encompasses prodrugs of the compounds of Formula I. The term “prodrug” as used herein means any covalently bonded carrier which releases the active parent drug of Formula I in vivo when such prodrug is administered to a mammalian subject. Prodrugs of the compounds of Formula I are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” means compounds that are rapidly transformed in viva to yield the parent compound of formula I, for example by hydrolysis in blood. Functional groups which may be rapidly transformed, by metabolic cleavage, in viva form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in viva, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ea., Elsevier, 1985; Methods in Enzymology, K. Widder et al, Ed., Academic Press, 42, p.309-396, 25 1985; A Textbook of Drug Design and Development, Krogsgaard-Larsen and H. Bundgaard, ea., Chapter 5; “Design and Applications of Prodrugs” p.113-191, 1991; Advanced Drug Delivery Reviews, H. Bundgard, 8, p.1-38, 1992; Journal of Pharmaceutical Sciences, 77, p. 285, 30 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ea., American Pharmaceutical Association and Pergamon Press, 1987, which are incorporated herein by reference. “Prodrugs” are considered to be any covalently bonded carriers which release the active parent drug of Formula I in vivo when such prodrug is administered to a mammalian subject. Prodrugs of the compounds of Formula I are prepared by modifying functional groups present in the compounds in such a way that the modifications are cleaved, either in routine manipulation or in vivo, to the parent compounds.

Prodrugs include compounds wherein hydroxy, amine, or sulfhydryl groups are bonded to any group that, when administered to a mammalian subject, cleaves to form a free hydroxyl, amino, or sulfhydryl group, respectively. Examples of Prodrugs include, but are not limited to, acetate, formate and benzoate derivatives of alcohol and amine functional groups in the compounds of Formula I, and the like.

Labeled compounds of the invention may be used for in vitro studies such as autoradiography of tissue sections or for in vivo methods, e.g. PET or SPECT scanning. Particularly, compounds of the invention are useful as standards and reagents in determining the ability of a potential pharmaceutical to bind to the CRF₁ receptor.

Compounds provided herein can have one or more asymmetric centers or planes, and all diastereomeric forms of the compound are included in the present invention.

Many geometric isomers of olefins, C═N double bonds, or the like can also be present in the compounds, and all such stable isomers are contemplated in the present invention. Compounds of the invention may be isolated in the optically pure form, for example, by resolution of the racemic form by conventional methods such as crystallization in the presence of a resolving agent, or chromatography, using, for example, a chiral HPLC column, or synthesized by an asymmetric synthesis route enabling the preparation of enantiomerically enriched material. The present invention encompasses all possible tautomers of the compounds represented by Formula I.

### DETAILED DESCRIPTION OF THE INVENTION

Examples of compounds of the invention are as follows:

(1R,2S)Acetic acid 1-[5-(6-dimethylamino-2-methyl-pyridin-3-yl)-3,6-diethyl-pyridin-2-ylamino]-indan-2-yl ester;

(1R,2S)Acetic acid 1-[5-(6-dimethylamino-2-methyl-pyridin-3-yl)-3,6-diethyl-pyridin-2-ylamino]-indan-2-yl ester toluene 4-sulfonic acid; and

(1R,2S)[5-(6-Dimethylamino-2-methyl-pyridin-3-yl)-3,6-diethyl-pyrazin-2-yl]-(2-ethoxy-indan-1-yl)-amine.

Compounds of the invention can be prepared using the reactions depicted in the following charts or variations thereof known to those skilled in the art. As illustrated in Chart A for an exemplary compound of the invention, the aminopyrazine A-II can be prepared from the suitably functionalized chloropyrazine A-I (see Chart B) by reaction with the appropriate heterocyclic or carbocyclic amine in the presence of a transition metal catalyst (e.g. palladium(II) acetate or tris(dibenzylideneacetone)dipalladium(0)), base (e.g. sodium or potassium tert-butoxide) in solvents such as but not limited to toluene, DMF, or dioxane. (for example see Buchwald, S. L. et al J. Org. Chem. 2000, 65, 1158. Acetate formation can be achieved by coupling with acetic anhydride or acetyl chloride in the presence of a base (see A-III). Ethers can be formed by coupling of an alkyl iodide to the sodium alkoxide of A-III. Halogenation of A-III can be accomplished by a number of methods well-known to those skilled in the art utilizing reagents such as N-chlorosuccinimide, N-bromosuccinimide, N-iodosuccinimide, bromine, iodine, pyridinium tribromide in solvents such as dichloromethane, acetic acid, DMF, etc, to give the halopyrazine A-IV. Formation of the claimed compounds is accomplished by a transition metal catalyzed coupling reaction with A-IV and an appropriate metalloaryl reagent such as aryl boronic acids (see for example Miyaura, N.; et al Chem. Rev. 1995, 95, 2457), aryl stannanes (see for example Mitchell, T. N. Synthesis 1992, 803), or aryl Grignards (see for example Miller, J. A. Tetrahedron Lett. 1998, 39, 7275).

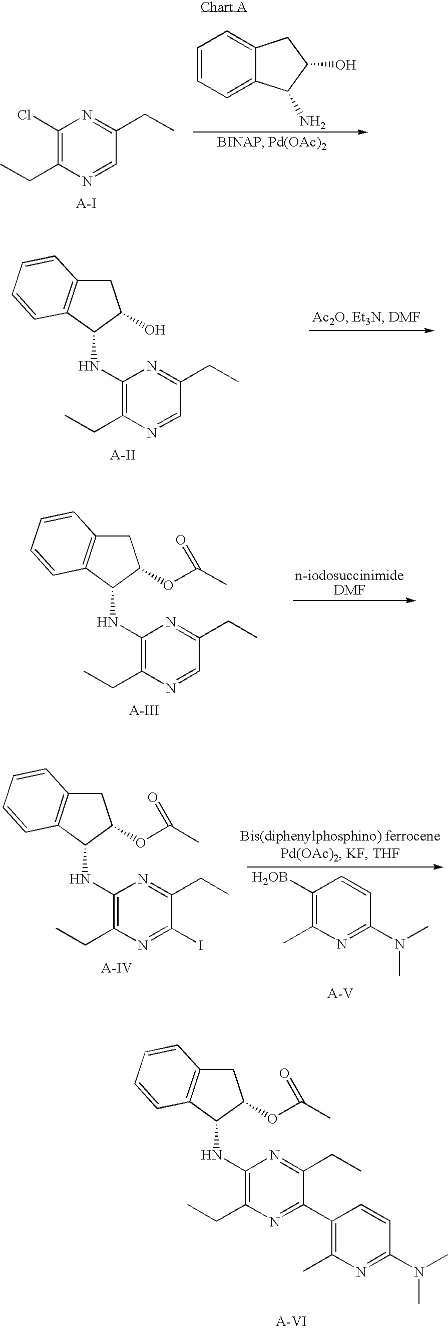

Chart B illustrates the preparation of mono chloro pyrazines, such as A-I. In the mono chloro pyrazines of Chart B, R₂ and R₂₂ can be the same C₁-C₆ alkyl groups, such as ethyl, or different C₁-C₆ alkyl groups by coupling the appropriate amino acids. The reaction sequence shown below follows that described in Chemical and Pharmaceutical Bulletin of Japan, 1979, 27, 2027.

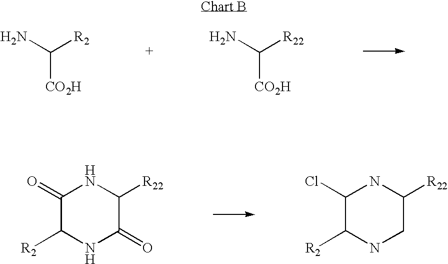

Chart C depicts the formation of an exemplary boronic acid coupling fragment. Boronic acids can be formed via metal halogen exchange or by palladium coupling methods known by those skilled in the art.

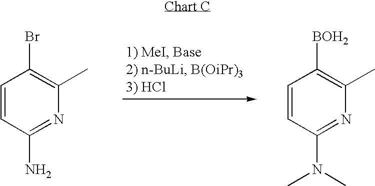

In addition to the conditions described hereinabove, compounds of the invention are useful for treating various disorders in a mammal, particularly in a human, such as social anxiety disorder; panic disorder; obsessive-compulsive disorder; anxiety with co-morbid depressive illness; affective disorder; anxiety; depression; irritable bowel syndrome; post-traumatic stress disorder; supranuclear palsy; immune suppression; gastrointestinal disease; anorexia nervosa or other feeding disorder; drug or alcohol withdrawal symptoms; substance abuse disorder (e.g., nicotine, cocaine, ethanol, opiates, or other drugs); inflammatory disorder; fertility problems; disorders the treatment of which can be effected or facilitated by antagonizing CRF₁ including but not limited to disorders induced or facilitated by CRF; a disorder selected from inflammatory disorders such as rheumatoid arthritis and osteoarthritis, pain, asthma, psoriasis and allergies; generalized anxiety disorder; panic, phobias, obsessive-compulsive disorder; post-traumatic stress disorder; sleep disorders induced by stress; pain perception such as fibromyalgia; mood disorders such as depression, including major depression, single episode depression, recurrent depression, child abuse induced depression, and postpartum depression; dysthemia; bipolar disorders; cyclothymia; fatigue syndrome; stress-induced headache; cancer, human immunodeficiency virus (HIV) infections; neurodegenerative diseases such as Alzheimer's disease, Parkinson's disease and Huntington's disease; skin disorders such as acne and psoriasis; gastrointestinal diseases such as ulcers, irritable bowel syndrome, Crohn's disease, spastic colon, diarrhea, and post operative ilius and colonic hypersensitivity associated by psychopathological disturbances or stress; hemorrhagic stress; stress-induced psychotic episodes; euthyroid sick syndrome; syndrome of inappropriate antidiarrhetic hormone (ADH); obesity; infertility; head traumas; spinal cord trauma; ischemic neuronal damage (e.g., cerebral ischemia such as cerebral hippocampal ischemia); excitotoxic neuronal damage; epilepsy; cardiovascular and hear related disorders including hypertension, tachycardia and congestive heart failure; stroke; immune dysfunctions including stress induced immune dysfunctions (e.g., stress induced fevers, porcine stress syndrome, bovine shipping fever, equine paroxysmal fibrillation, and dysfunctions induced by confinement in chickens, sheering stress in sheep or human-animal interaction related stress in dogs); muscular spasms; urinary incontinence; senile dementia of the Alzheimer's type; multiinfarct dementia; amyotrophic lateral sclerosis; chemical dependencies and addictions (e.g., dependences on alcohol, cocaine, heroin, benzodiazepines, or other drugs); osteoporosis; psychosocial dwarfism and hypoglycemia.

A compound of this invention can be administered to treat the conditions described herein in a mammal or human by means that produce contact of the active agent with the agent's site of action in the body of the mammal or human. The compounds can be administered by any conventional means available for use in conjunction with pharmaceuticals either as individual therapeutic agent or in combination of therapeutic agents. It can be administered alone, but will generally be administered with a pharmaceutical carrier selected on the basis of the chosen route of administration and standard pharmaceutical practice.

The dosage administered will vary depending on the use and known factors such as pharmacodynamic character of the particular agent, and its mode and route of administration; the recipient's age, weight, and health; nature and extent of symptoms; kind of concurrent treatment; frequency of treatment; and desired effect.

For use in the treatment of the diseases or conditions described herein, a compound of this invention can be orally administered at a dosage of the active ingredient of 0.002 to 200 mg/kg of body weight. Ordinarily, a dose of 0.01 to 10 mg/kg in divided doses one to four times a day, or in sustained release formulation will be effective in obtaining the desired pharmacological effect.

The active ingredient can be administered orally in solid dosage forms, such as capsules, tablets and powders; or in liquid forms such as elixirs, syrups, and/or suspensions. The compounds of this invention can also be administered parenterally in sterile liquid dose formulations. Dosage forms (compositions) suitable for administration contain from about 1 mg to about 100 mg of active ingredient per unit. In these pharmaceutical compositions, the active ingredient will ordinarily be present in an amount of about 0.5 to 95% by weight based on the total weight of the composition.

The compounds of this invention may also be used as reagents or standards in the biochemical study of neurological function, dysfunction, and disease.

### PREPARATIONS AND EXAMPLES

The invention is illustrated further by the following examples and preparations, which are not to be construed as limiting the invention in scope or spirit to the specific procedures described in them.

### Example A

### CRF₁ Receptor Binding Assay for the Evaluation of Biological Activity

The following is a description of the isolation of rat brain membranes for use in the standard binding assay as well as a description of the binding assay itself. It is based on a modified protocol described by De Souza (De Souza, 1987).

To prepare brain membranes for binding assays, rat frontal cortex is homogenized in 10 mL of ice cold tissue buffer (50 mM HEPES buffer pH 7.0, containing 10 mM MgCl₂, 2 mM EGTA, 1 μg/ml aprotinin, 1 μg/ml leupeptin and 1 μg/ml pepstatin). The homogenate is centrifuged at 48,000×g for 10 min. and the resulting pellet rehomogenized in 10 mL of tissue buffer. Following an additional centrifugation at 48,000×g for 10 min., the pellet is resuspended to a protein concentration of 300 μg/mL.

Binding assays are performed in 96 well plates at a final volume of 300 μL. The assays are initiated by the addition of 150 μL membrane suspension to 150 μL of assay buffer containing ¹²⁵I-ovine-CRF (final concentration 150 pM) and various concentrations of inhibitors. The assay buffer is the same as described above for membrane preparation with the addition of 0.1% ovalbumin and 0.15 mM bacitracin. Radioligand binding is terminated after 2 hours at room temperature by filtration through Packard GF/C unifilter plates (presoaked with 0.3% polyethyleneimine) using a Packard cell harvestor. Filters are washed three times with ice cold phosphate buffered saline pH 7.0 containing 0.01% Triton X-100. Filters are assessed for radioactivity in a Packard TopCount. Nonspecific binding is determined in the presence of excess (10 μM) α-helical CRF.

Alternatively, tissues and cells that naturally express CRF receptors, such as IMR-32 human neuroblastoma cells (ATCC; Hogg et al., 1996), can be employed in binding assays analogous to those described above.

IC₅₀ values are calculated using standard methods known in the art, such as with the non-linear curve fitting program RS/1 (BBN Software Products Corp., Cambridge, Mass.). A compound is considered to be active if it has an IC₅₀ value of less than about 10 micromolar (μM) for the inhibition of CRF₁ receptors. The binding affinity of the compounds of Formula I expressed as IC₅₀ values generally ranges from about 0.5 nanomolar to about 10 micromolar. Preferred compounds of Formula I exhibit IC₅₀ of 1 micromolar or less, more preferred compounds of Formula I exhibit IC₅₀ of less than 100 nanomolar or less, still more preferred compounds of Formula I exhibit IC₅₀ of less than 10 nanomolar or less.

### Example B

### Inhibition of CRF-Stimulated Adenylate Cyclase Activity

Inhibition of CRF-stimulated adenylate cyclase activity can be performed as previously described [G. Battaglia et al., Synapse 1:572 (1987)]. Briefly, assays are carried out at 37° C. for 10 min in 200 mL of buffer containing 100 mM Tris-HCl (pH 7.4 at 37° C.), 10 mM MgCl₂, 0.4 mM EGTA, 0.1% BSA, 1 mM isobutylmethylxanthine (IBMX), 250 units/mL phosphocreatine kinase, 5 mM creatine phosphate, 100 mM guanosine 5′-triphosphate, 100 nM o-CRF, antagonist peptides (various concentrations) and 0.8 mg original wet weight tissue (approximately 40-60 mg protein). Reactions are initiated by the addition of 1 mM ATP/[³²P]ATP (approximately 2-4 mCi/tube) and terminated by the addition of 100 mL of 50 mM Tris-HCl, 45 mM ATP and 2% sodium dodecyl sulfate. In order to monitor the recovery of cAMP, 1 mL of [³H]cAMP (approximately 40,000 dpm) is added to each tube prior to separation. The separation of [³²P]cAMP from [³²P]ATP is performed by sequential elution over Dowex and alumina columns.

Alternatively, adenylate cyclase activity can be assessed in a 96-well format utilizing the Adenylyl Cyclase Activation FlashPlate Assay from NEN Life Sciences according to the protocols provided. Briefly, a fixed amount of radiolabeled cAMP is added to 96-well plates that are precoated with anti-cyclic AMP antibody. Cells or tissues are added and stimulated in the presence or absence of inhibitors. Unlabeled cAMP produced by the cells will displace the radiolabeled cAMP from the antibody. The bound radiolabeled cAMP produces a light signal that can be detected using a microplate scintillation counter such as the Packard TopCount. Increasing amounts of unlabeled cAMP results in a decrease of detectable signal over a set incubation time (2-24 hours).

### EXAMPLES

### Preparation 1

### (1R,2S)-1-(3,6-Diethyl-pyrazin-2-ylamino)-indan2-ol

To a nitrogen purged 200 Liter glass lined reactor was added (1R,2S)-(+)-cis-1-amino-2-indanol (2.5 kg, 16.1 moles, 1.5 eq), palladium (II) acetate (72 g, 0.3 moles, 3 mole % ), 2,2′-bis(diphenylphosphino)-1,1′-binapthyl (200 g, 0.3 moles, 3 mole % ) and cesium carbonate (7.0 kg, 21.5 moles, 2.0 eq) followed by toluene (65 L, drum stock). To the stirring white suspension was added 3-Chloro-2,5-diethyl-pyrazine (1.83 kg, 10.7 moles, 1.0 eq) at room temperature and the contents were heated to reflux (110° C.) for 2 h, at which time the reaction was judged complete by HPLC (4 drops of reaction mixture quenched into water and then extracted into 1 mL MTBE, remove solvent and dilute with 1.5 mL CH₃CN/water). To the ambient reaction mixture was added methyl-t-butyl ether (45 L, drum stock) and water (45 L) and the layers separated. The organic layer was washed a second time with water (45 L) then extracted with methyl-t-butyl ether (45 L, drum stock). The combined organic layers were then concentrated under vacuum to a minimum volume. Dimethyl formamide (4 gal, E&M Science) was added and the resultant black solution was transferred into a 20-L bottle. A yield for (1R,2S)-1-(3,6-Diethyl-pyrazin-2-ylamino)-indan2-ol using quantitative HPLC (2.27 kg, 73%) was determined. This material was used without further purification. HPLC retention time of the title compound is 2.1 min. Column 150 mm×4.6 mm, Luna 5μphenyl-hexyl; 50/50 CH₃CN/water+0.1% TFA with gradient to 75/25+0.1% CH₃CN/water+0.1% TFA.IR (diffuse reflectance) 3435, 3241, 2962, 2935, 2912, 2873, 1581, 1547, 1500, 1453, 1184, 1163, 1047, 744, 733 cm⁻¹; OAMS supporting ions at: ESI+384.0; MS (Cl ) m/z 284 (MH⁺); HRMS (FAB) calcd for C₁₇H₂₁N₃O+H₁ 284.1763, found 284.1754. [□[²⁵_{D}=12(c 0.55, methylene chloride); Anal. Calcd for C₁₇H₂₁N₃O: C, 72.06; H, 7.47; N, 14.83. Found: C, 72.15; H, 7.53 N, 14.42.

### Preparation 2

### (1R,2S)Acetic Acid 1-(3,6-diethyl-5-iodo-pyrazin-2-ylamino)-indan-2-yl ester

To a nitrogen purged 1200 L glass lined reactor was added (1R,2S)-1-(3,6-Diethyl-pyrazin-2-ylamino)-indan2-ol (25 kg, 86.1 moles, 1.0 eq), 4-dimethylamino pyridine (1.0 kg, 8.6 moles, 10mole %) and tetrahydrofuran (139 L, drum stock) followed by triethylamine (18 kg, 177.9 moles, 2.1 eq). To this solution, acetic anhydride (10.6 kg, 103.8 moles, 1.2 eq) was added while maintaining an internal temperature of less than 30° C. After stirring for 3 h at 20-25° C., HPLC (3 drops quenched into 1.0 mL methanol then diluted with 0.5 mL water) showed incomplete reaction. Additional acetic anhydride (2.4 kg, 23.8 moles, 0.3 eq) was added and the contents were stirred for 1 h then re-assayed and judged complete. Methanol (6.3 kg, 197.2) moles was added to consume excess acetic anhydride and stirred for 1 h after which, the mixture was diluted with methyl-t-butyl ether (200 L) and water (200 L) containing citric acid (23.0 kg, 119.7 moles). The phases were separated and the aqueous layer was extracted with methyl-t-butyl ether (100 L). the combined organic phases were washed with 1N aqueous sodium hydroxide (200 L) and water (2×100 L). The combined organics were distilled under vacuum to less than 75 L at which time dimethylformamide (150 L, drum stock) was added and the concentration continued to a tank volume of ˜160 L. This solution was added to a second 1200 L glass lined reactor containing N-iodosuccinimide (30.0 kg, 133.3 moles, 1.5 eq) and then heated to 55° C. for 3 h at which time the reaction was judged complete by HPLC (3 drops of reaction mixture quenched into water and then extracted into 1 mL MTBE, remove solvent and dilute with 1.5 mL CH₃CN/water). The ambient mixture was diluted with methyl-t-butyl ether (200 L) and treated with water (200 L) containing sodium thiosulfate pentahydrate (22.6 kg, 91 moles). The layers were separated and the aqueous layer was extracted with methyl-t-butyl ether (100 L). The combined organic layers were washed with water (3×100 L) and then distilled to a low volume under vacuum to afford crude (1R,2S)Acetic Acid 1-(3,6-diethyl-5-iodo-pyrazin-2-ylamino)-indan-2-yl ester. Purification was done over silica (500 kg) eluting with 20/80 EtOAc/octane collecting 200-L fractions. Concentration of the appropriate column fractions while adding octane gave a suspension that was cooled to 0° C., filtered and washed with octane, then dried with 40° C. nitrogen to afford 31.1 kg (80%) of the title compound as a white solid. ¹H NMR (400 MHz, DMSO-d₆) δ 8 7.28 (m, 4 H), 6.66 (d, J=9 Hz, 1 H), 5.80 (m, 1 H), 5.68 (m, 1 H), 3.29 (m, 1 H), 3.01 (d, J=17 Hz, 1 H), 2.69 (m, 4 H), 1.88 (s, 3 H), 1.15 (m, 6 H); ¹³C NMR (DMSO-d₆) δ 169.72, 153.75, 151.01, 143.73, 141.24, 139.89, 127.80, 126.75, 124.72, 124.39, 100.66, 74.33, 57.01, 36.82, 31.04, 24.71, 20.86, 12.60, 11.17.

### Preparation 3

### (5-Bromo-6-methyl-pyridin-2-yl)-dimethyl-amine

To a solution of 5-Bromo-6-methyl-pyridin-2-ylamine (4 g, 0.021 mole) in tetrahydrofuran (105 mL) was added sodium hydride (60%, 1.2 eq. 1 g). After 30 min, iodomethane (1.56 ml, 1.2 eq.) was added. After an additional 24 h, sodium hydride (60%, 1.2 eq. 1 g). and iodomethane (1.56 ml, 1.2 eq.) were added. The reaction mixture stirred 72 h, and was poured into 1N NaOH, extracted with ethyl ether, dried magnesium sulfated, filtered and concentrated. MPLC biotage chromatography eluting with 2-10% ethyl acetate/hexane provided the title compound as an oil. (4.31 g, 96%). ¹H NMR (400 MHz, CDCl₃) δ 7.45 (d, J=8.7 Hz, 1 H), 6.20 (d, J=8.7 Hz, 1 H), 3.01 (s, 6H), 2.46 (s, 3H).

### Preparation 4

### (5-Boronic Acid-6-methyl-pyridin-2-yl)-dimethyl-amine

To a solution of (5-Bromo-6-methyl-pyridin-2-yl)-dimethyl-amine (1.0 g, 0.0046 mole) in tetrahydrofuran (1.6 ml)/toluene (6.6 ml) was added n-BuLi (2.24 ml of 2.5M) dropwise under nitrogen atmosphere at −78° C. After 30 min, triisopropyl borate (1.28 ml) was added dropwise. After 30 min, the reaction mixture was warmed to ambient temperature and stirred 30 min followed by the addition of 7 mL of 1 N HCl. The reaction mixture stirred 1H and quenched to pH8 with 1 N NaOH. Extraction with ethyl acetate, drying with magnesium sulfated and concentration provided a white solid. Trituration with hexane and filtration provided the title compound as a white solid 550 mg (65%) (400 MHz, DMSO) δ 7.90 (m, 1 H), 6.45 (m, 1 H), 3.01 (s, 6H), 2.63 (s, 3H).

### Example 1

### (1R,2S)Acetic acid 1-[5-(6-dimethylamino-2-methyl-pyridin-3-yl)-3,6-diethyl-pyrazin-2-ylamino]-indan-2-yl ester

To a clean a dry 1 Liter 3 necked round bottom flask, equipped with an overhead stirrer, equipped with a nitrogen inlet tube, and reflux condenser, was charged Tetrahydrofuran (8.60 moles; 700 mL; 620 g), (5-Boronic Acid-6-methyl-pyridin-2-yl)-dimethyl-amine (1.00 equiv [Limiting Reagent]; 194 mmoles; 35.0 g), (1R,2S)Acetic Acid 1-(3,6-diethyl-5-iodo-pyrazin-2-ylamino)-indan-2-yl ester (0.500 equiv; 97.2 mmoles; 43.9 g) Pd (OAc)₂ (0.0200 equiv; 3.89 mmoles; 873 mg), 1,1′-Bis(diphenylphosphino)ferrocene (0.0200 equiv; 3.89 mmoles; 2.16 g), Potassium Hydrogen Fluoride, 99-100-wt/wt % (4.00 equiv; 778 mmoles; 61.0 g). The reaction mixture was heated to 60 C and held for 18 hrs. The reaction was then cooled to room temperature, filtered and the product was isolated via chromatography (20% METB/Hexane). 42 gm of the desired product was recovered. This was used without further purification. (Low melting solid) ¹H NMR (400 MHz, CDCl₃) δ 7.37 (m, 1 H), 7.28 (m, 4 H), 6.40 (d, J=8.7 Hz, 1 H), 6.05 (m, 1 H), 5.72 (m, 1H), 4.82 (d, J=9.1 Hz, 1 H), 3.33 (dd, J=17.0, 5.0 Hz, 1 H), 3.08 (s, 6 H), 2.05 (m, 1 H), 2.67 (q, J=7.5 Hz, 2H). 2.49 (q, J=7.5 Hz, 2H), 2.23 (s, 3H), 1.94 (s, 3H), 1.27 (m, 3H), 1.12 (t, J=7.5 Hz, 3H). (Parent M⁺H m/z=460.4).

### Example 2

### (1R,2S)Acetic acid 1-[5-(6-dimethylamino-2-methyl-pyridin-3-yl)-3,6-diethyl-pyrazin-2-ylamino]-indan-2-yl ester toluene 4-sulfonic acid

To a clean and dry 2-Methyl THF rinsed round bottom flask was charged, 650 ml of 2-Methyl THF, 65 gm of (1R,2S)Acetic acid 1-[5-(6-dimethylamino-2-methyl-pyridin-3-yl)-3,6-diethyl-pyrazin-2-ylamino]-indan2-yl ester. This solution was filtered to a spec-free/2-methyl THF rinsed 2 L round bottom flask. To this was added via a filtration a solution of 150 ml of 2-Methyl THF and 34.4 gm of p-Toluenesulfonic acid monohydrate. The salt solution is heated to 60 C and allowed to cool to room temperature. The product is allowed to granulate at ambient temperature and isolated via filtration washed with filtered 2-Methyl THF and dried in a vacuum oven overnight at 45 C. The produce (79.2 gm, 89% yield) was consistent for the desired structure and powder x-ray matched the desired polymorph form. 1H NMR (400 MHz, CDCl3) 67 7.80 (d, J=8.3 Hz, 2 H), 7.67 (d, J=9.5 Hz, 1H), 7.34 (m, 1H), 7.29 (m, 3H), 7.15 (d, J=8.7 Hz, 2 H), 6.72 (d, J=9.1 Hz, 1 H), 6.03 (m, 1H), 5.72 (m, 1H), 4.97 (d, J=9.1 Hz, 1 H), 3.39 (s, 6H), 3.34 (dd, J=17.4, 5.4 Hz, 1 H), 3.09 (d, J=17.0 Hz, 1 H), 2.63 (m, 2 H), 2.57 (s, 3 H), 2.42 (q, J=7.5 Hz, 2 H), 2.32 (s, 3H), 1.96 (s, 3H), 1.27 (t, J=7.5 Hz, 3H), 1.15 (t, J=7.5 Hz, 3H); MS: (Parent M+H m/z=460.1); Anal. Calcd for C34H41N505S: C, 64.64; H, 6.54; N, 11.08; S, 5.07. Found: C, 64.27; H, 6.57; N, 10.94; S, 5.41.

### Preparation 5

### (1R,2S)1-[5-(6-Dimethylamino-2-methyl-pyridin-3-yl)-3,6-diethyl-pyrazin-2-ylamino]-indan-2-ol

To a solution of (1R,2S)1-(3,6-Diethyl-5-iodo-pyrazin-2ylamino)-indan-2-ol (1 g) in benzene (20 mL) was added (5-Boronic Acid-6-methyl-pyridin-2-yl)-dimethyl-amine

(880 mg, 2 eq.), dichloropalladium ditriphenylphosphine (171 mg, 0.1 eq.) and 2N sodium carbonate solution (4 mL) and the reaction mixture was heated at 75° C. for 18 h. The reaction mixture was cooled to ambient temperature, poured into saturated bicarbonate and extracted 2× ethyl acetate. The organic layer was dried with magnesium sulfate, filtered and concentrated. Purification via Biotage MPLC eluting with 20-40% ethyl acetate/hexane provided the title compound (355 mg, 36%). ¹H NMR (400 MHz, CDCl₃) δ 7.23 (m, 5 H), 6.40 (d, J=8.3 Hz, 1 H), 6.57 (t, J=5.4 Hz, 1H), 4.80 (m, 2 H), 3.21 (m, 2 H), 3.08 (s, 6 H), 2.70 (q, J=7.5 Hz, 2 H), 2.51 (q, J=7.5 Hz, 2H), 2.23 (s, 3H), 1.28 (t, J=7.5 Hz, 3H), 1.12 (t, J=7.5 Hz, 3H); MS: (Parent M^{+H} m/z=418.3).

### Example 3

### (1R,2S)[5-(6-Dimethylamino-2-methyl-pyridin-3-yl)-3,6-diethyl-pyrazin-2-yl]-(2-ethoxy-indan-1-yl)-amine

To a solution of (1R,2S)1-[5-(6-Dimethylamino-2-methyl-pyridin-3-yl)-3,6-diethyl-2-ylamino]-indan-2-ol (93 mg) in dimethyl formamide (2.2 mL) at 0° C. was added sodium hydride (11 mg, 1.2 eq.) under N₂. After 5 min, iodo ethane (1.2 eq.) was added. After 2 h, the reaction mixture was poured into saturated sodium bicarbonate, extracted with methylene chloride, dried magnesium sulfate, filtered and concentrated. Purification via Biotage MPLC eluting with 5-20% ethyl acetate/hexane provided the title compound (61 mg). ¹H NMR (400 MHz, CDCl₃) δ 7.43 (d, J=6.6 Hz, 1H), 7.25 (m, 1 H), 7.23 (m, 3 H), 6.40 (d, J=8.3 Hz, 1 H), 5.79 (m, 1 H), 5.26 (d, J=7.9 Hz, 1 H), 4.35 (m, 1 H), 3.66 (m, 1 H), 6.40 (d, 1H), 3.10 (m, 2H), 3.09 (s, 6 H), 2.70 (q, J=7.5 Hz, 2 H), 2.50 (q, J=7.5 Hz, 2H), 2.24 (s, 3H), 1.28 (t, J=7.5 Hz, 3H), 1.12 (m,6 H); MS: (Parent M⁺H m/z=446.3).

The value of Kᵢ, the binding constant to the CRF₁ receptor, was measured for exemplary compounds of the invention. The compound of Example 1, (1R,2S)Acetic acid 1-[5-(6-dimethylamino-2-methyl-pyridin-3-yl)-3,6-diethyl-pyrazin-2-ylamino]-indan-2-yl ester, was found to have a Kᵢ of 19 nM. The compound of Example 3, (1R,2S)[5-(6-Dimethylamino-2-methyl-pyridin-3-yl)-3,6-diethyl-pyrazin-2-yl]-(2-ethoxy-indan-1-yl )-amine, was found to have a Kᵢ of 13 nM. These results provide strong evidence in favor of the capability of the compounds of the invention to act as CRF₁ receptor antagonists.

The specific embodiments disclosed herein are intended as illustrative of aspects of the invention and are not intended to limit the scope of the invention in any way. Any equivalent embodiments are intended to be within the scope of this invention. Various modifications of the invention in addition to those shown and described herein will be apparent to those skilled in the art from the foregoing description. Such modifications are also intended to fall within the scope of the appended claims.

We claim:

1. A compound of formula I 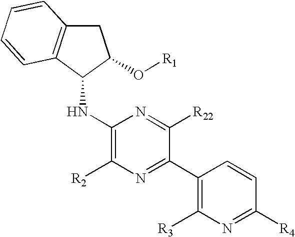
or a pharmaceutically acceptable salt thereof, wherein
R₁ is C₁-C₆ alkyl, C₁-C₆ alkenyl, C₁-C₆ alkynyl, C(O)C₁-C₆ alkyl, C(O)C₁-C₆ alkenyl or C(O)C₁-C₆ alkynyl;
R₂ is C₁-C₆ alkyl, C₁-C₆ alkenyl, or C₁-C₆ alkynyl;
R₂₂ is C₁-C₆ alkyl, C₁-C₆ alkenyl, or C₁-C₆ alkynyl;
R₃ is C₁-C₆ alkyl, C₁-C₆ alkenyl, C₁-C₆ alkynyl, halogen, OC₁-C₆ alkyl, OC₁-C₆ alkenyl, or O₁-C₆ alkynyl;
R₄ is C₁-C₆ alkyl, C₁-C₆ alkenyl, C₁-C₆ alkynyl, halogen, OC₁-C₆ alkyl, OC₁-C₆ alkenyl, OC₁-C₆ alkynyl or NR₅Rᵣ;
R₅ is hydrogen, C₁-C₆ alkyl, C₁-C₆ alkenyl, or C₁-C₆ alkynyl;
and
R₆ is hydrogen, C₁-C₆ alkyl, C₁-C₆ alkenyl, or C₁-C₆ alkynyl.

2. The compound of claim 1, wherein R₁ is ethyl or C(O)CH₃.

3. The compound of claim 1, wherein R₂ is ethyl and R₂₂ is ethyl.

4. The compound of claim 1, wherein R₃ is C₁-C₆ alkyl, C₁-C₆ alkenyl, or C₁-C₆ alkynyl.

5. The compound of claim 1, wherein R₄ is NR₅R₆.

6. The compound of claim 5, wherein R₃ is C₁-C₆ alkyl, C₁-C₆ alkenyl, or C₁-C₆ alkynyl.

7. The compound of claim 6, wherein R₃ is methyl and R₄ is N(CH₃)₂.

8. A compound selected from the group consisting of
(1R,2S)Acetic acid 1-[5-(6-dimethylamino-2-methyl-pyridin-3-yl)-3,6-diethyl-pyrazin-2-ylamino]-indan-2-yl ester;
(1R,2S)Acetic acid 1-[5-(6-dimethylamino-2-methyl-pyridin-3-yl)-3,6-diethyl-pyrazin-2-ylamino]-indan-2-yl ester toluene 4-sulfonic acid; and
(1R,2S)[5-(6-Dimethylamino-2-methyl-pyridin-3-yl)-3,6-diethyl-pyrazin-2-yl]-(2-ethoxy-indan-1-yl)-amine.

9. A pharmaceutical composition comprising a pharmaceutically acceptable carrier and a compound according to claim 1.

10. The pharmaceutical composition of claim 9, wherein the compound according to claim 1 is present in an amount that is therapeutically effective for the treatment of a disorder or disease that is associated with CRF₁ receptors, or a disorder the treatment of which can be effected or facilitated by antagonizing CRF₁, in a mammal.

11. A method for the treatment of a disorder or disease that are associated with CRF₁ receptors, or a disorder the treatment of which can be effected or facilitated by antagonizing CRF₁ in a mammal, the method comprising administering to the mammal a compound according to claim 1.

12. A method for the treatment of a disorder selected from the group consisting of generalized anxiety disorder, social anxiety disorder, panic disorder, obsessive-compulsive disorder, anxiety with co-morbid depressive illness, affective disorder, anxiety, eating disorders, bipolar disorder and depression in a mammal, the method comprising administering to the mammal a compound according to claim 1.

13. A method of treating a disorder manifesting hypersecretion of CRF in a mammal, comprising administering to the mammal a therapeutically effective amount of a compound according to claim 1.

14. A method for screening for ligands for CRF₁ receptors, which method comprises: a) carrying out a competitive binding assay with CRF₁ receptors, a compound according to claim 1 which is labeled with a detectable label, and a candidate ligand; and b) determining the ability of said candidate ligand to displace said labeled compound.

15. A method for detecting CRF receptors in tissue comprising: a) contacting a compound according to claim 1 which is labeled with a detectable label, with a tissue, under conditions that permit binding of the compound to the tissue; and b) detecting the labeled compound bound to the tissue.

16. A method of inhibiting the binding of CRF to a CRF₁ receptor, comprising contacting a compound according to claim 1 with a solution comprising cells expressing the CRF₁ receptor, wherein the compound is present in the solution at a concentration sufficient to inhibit the binding of CRF to the CRF₁ receptor.

17. A method of reducing the level of CRF binding in vitro to cells expressing the CRF₁ receptor, comprising contacting a compound according to claim 1 with a solution comprising the cells, wherein the compound is present in the solution at a concentration sufficient to reduce levels of CRF binding to the cells in vitro.

18. An article of manufacture comprising: a) a packaging material; b) a compound according to claim 1; and c) a label or package insert contained within said packaging material indicating that said compound is effective for treating a disorder or disease that is associated with CRF₁ receptors, or a disorder the treatment of which can be effected or facilitated by antagonizing CRF₁, in a mammal.

19. A compound of claim 1 wherein the compound exhibits an IC₅₀ for CRF binding of 1 micromolar or less in a standard CRF binding assay.

20. A compound of claim 18 wherein the compound exhibits an IC₅₀ for CRF binding of 100 nanomolar or less.

21. A compound of claim 18 wherein the compound exhibits an IC₅₀ for CRF binding of 10 nanomolar or less.
